(19) United States
(12) Patent Application Publication
(10) Pub. No.: US 2019/0239722 A1
(43) Pub. Date: Aug. 8, 2019

(54) CONTROLLER AND INSERTION APPARATUS

(71) Applicant: OLYMPUS CORPORATION, Tokyo (JP)

(72) Inventors: Yoshitaka Umemoto, Hachioji-shi (JP); Takashi Suzuki, Hino-shi (JP); Yasuaki Natori, Akishima-shi (JP); Takashi Yamashita, Hachioji-shi (JP); Fumiyuki Onoda, Tama-shi (JP)

(73) Assignee: OLYMPUS CORPORATION, Tokyo (JP)

(21) Appl. No.: 16/386,365

(22) Filed: Apr. 17, 2019

Related U.S. Application Data
(63) Continuation of application No. PCT/JP2017/035978, filed on Oct. 3, 2017.

(30) Foreign Application Priority Data
Oct. 21, 2016 (JP) 2016-206846

(51) Int. Cl. A61B 1/00; A61B 1/04; G02B 23/24

(52) U.S. Cl. CPC A61B 1/0016; G02B 23/2476; A61B 1/04; A61B 1/00006

(57) ABSTRACT

A controller is configured to control an operation of a self-propelled mechanism provided in an elongated insertion section of an endoscope. The controller includes a circuit configured to: acquire a speed value corresponding to a rotation speed of a motor of the self-propelled mechanism; acquire a torque value corresponding to a torque generated by the motor; control rotation of the motor by switching between at least two of speed control that controls rotation of the motor based on the speed value and torque control that controls rotation of the motor based on the torque value; and stop the rotation of the motor if the torque value has exceeded a predetermined threshold value, perform the torque control when the rotation of the motor is resumed.

### CROSS-REFERENCE TO RELATED APPLICATIONS

This application is a Continuation Application of PCT Application No. PCT/JP2017/035978, filed Oct. 3, 2017 and based upon and claiming the benefit of priority from prior Japanese Patent Application No. 2016-206846, filed Oct. 21, 2016, the entire contents of all of which are incorporated herein by reference.

### BACKGROUND OF THE INVENTION

### 1. Field of the Invention

The present invention relates to a controller configured to control the operation of a self-propelled mechanism of an endoscope, and an insertion apparatus.

### 2. Description of the Related Art

In an insertion apparatus including an endoscope, the endoscope includes an insertion section to be inserted into, for example, a body cavity. Among such insertion apparatuses, a self-propelled insertion apparatus is known, in which a self-propelled mechanism is provided in the insertion section. For example, Jpn. Pat. Appln. KOKAI Publication No. 2007-185389 discloses a technique relating to a rotary, self-propelled endoscope. In this rotary, self-propelled endoscope apparatus, a rotating cylindrical body with a spiral-shaped fin formed on an outer circumferential face of an insertion section is provided. When such a rotating cylindrical body rotates, the fin formed on the rotating cylindrical body contacts an inner wall of the body cavity, thus generating a propulsion force. By this propulsion force, the insertion section is propelled by itself in the direction of insertion or the direction of removal. Also, Jpn. Pat. Appln. KOKAI Publication No. 2007-185389 discloses stopping a driving source of a rotating cylindrical body based on an increase in load applied to the rotating cylindrical body, and predicting and avoiding stop of the driving source of the rotating cylindrical body based on increase in load, thus improving the working efficiency.

### BRIEF SUMMARY OF THE INVENTION

According to an aspect of the present invention, a controller is configured to control an operation of a self-propelled mechanism provided in an elongated insertion section of an endoscope. The self-propelled mechanism includes a motor configured to generate a force that causes the insertion section to be inserted into or removed from a subject. The controller includes an input device to which an input is provided to switch a control mode and at least one circuit configured to: acquire a speed value corresponding to a rotation speed of the motor; acquire a torque value corresponding to a torque generated by the motor; control rotation of the motor by switching the control mode, the control mode including at least two of speed control that controls rotation of the motor based on the speed value and torque control that controls rotation of the motor based on the torque value; and stop the rotation of the motor if the torque value has exceeded a predetermined threshold value, perform the torque control when the rotation of the motor is resumed, and change the control mode from the torque control to the speed control when the input is provided after the control mode has been changed from the speed control to the torque control.

According to another aspect of the present invention, an insertion apparatus includes the above mentioned controller and the endoscope.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view of a configuration example of an insertion apparatus according to a first embodiment.

FIG. 2 is a flowchart showing an example of an operation of the insertion apparatus according to the first embodiment.

FIG. 3 is a diagram illustrating an example of an operation according to the first embodiment.

FIG. 4 is a flowchart showing an example of an operation of the insertion apparatus according to a second embodiment.

FIG. 5 is a diagram illustrating an example of an operation according to the second embodiment.

### DETAILED DESCRIPTION OF THE INVENTION

### First Embodiment

A first embodiment of the present invention will be described with reference to the accompanying drawings. An insertion apparatus according to the present embodiment is a system relating to an endoscope comprising a rotary, self-propelled mechanism. In output control of the self-propelled mechanism according to the present embodiment, a speed control mode in which the rotation speed is adjusted to a predetermined value and a torque control mode in which the rotation torque is adjusted to a predetermined value, are selectively used.

<Configuration of Insertion Apparatus>

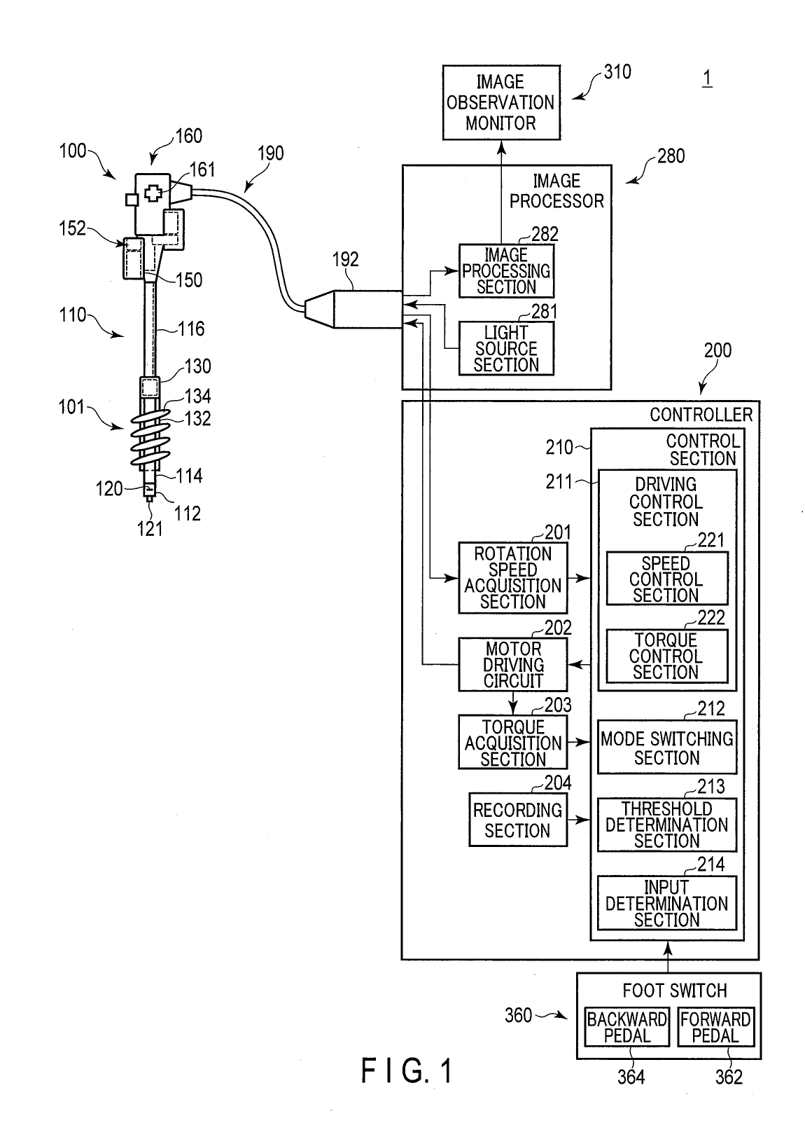

FIG. 1 schematically shows a configuration of an endoscope system as an example of an insertion apparatus according to an embodiment of the present invention. As shown in the figure, an insertion apparatus 1 includes an endoscope 100, a controller 200, an image processor 280, an image observation monitor 310, and a foot switch 360.

The endoscope 100 is a rotary, self-propelled endoscope. The endoscope 100 includes an insertion section 110. The insertion section 110 is in an elongated shape, and is configured to be inserted into a living body, for example, which is a subject to be inserted. The endoscope 100 includes a control unit 160 for performing various manipulations on the endoscope 100. The control unit 160 is held by a user. Herein, a side distal to the insertion section 110 will be referred to as a distal side. Also, a side on which the control unit 160 of the insertion section 110 is provided will be referred to as a proximal side. A direction going from the distal side toward the proximal side of the insertion section 110 will be referred to as a longitudinal direction. A control unit 160 and an image processor 280 of the endoscope 100 are connected through a universal cable 190. A connector 192 is provided at an end portion of the universal cable 190, and the connection between the image processor 280 and the universal cable 190 is provided by the connector 192. The image processor 280 and the controller 200 are connected, and the endoscope 100 and the controller 200 are connected through the image processor 280.

The insertion section 110 includes a rigid distal end 112, a bending section 114, and an insertion tube 116. The rigid distal end 112 is a section at the extremity of the distal end of the insertion section 110, and is configured not to be bent. The bending section 114 is a section formed on the proximal side of the rigid distal end 112, and is configured to be actively bent in response to a manipulation on a manipulation section 161 provided in the control unit 160. The insertion tube 116 is a section formed on the proximal side of the bending section 114, and is configured to be passively bent by an external force.

The rigid distal end 112 includes an imaging element 120 and an illumination lens 121. The imaging element 120 generates an image signal based on a subject image on the distal side of, for example, the insertion section 110. The image signal generated by the imaging element 120 is transmitted to the image processor 280 through a signal line for image signals (not shown in the drawings) passing through the insertion section 110 and the universal cable 190. The illumination lens 121 diffuses light guided from the image processor 280 through an optical fiber (not shown in the drawings) passing through the insertion section 110 and the universal cable 190, and emits the diffused light.

The insertion apparatus 1 according to the present embodiment comprises a self-propelled mechanism 101. That is, a rotating section 130 for transmitting a driving force of the motor 150 provided in the control unit 160 is attached to the insertion tube 116 of the insertion section 110. Also, a power spiral tube 132, which is a rotating cylindrical body, is attached to a distal side of the rotating section 130. The power spiral tube 132 is formed in a cylindrical form with an elastic material such as rubber, resin, etc., and is rotatably attached to the insertion tube 116 around its longitudinal axis. A spiral-shaped fin 134 is provided on an outer circumferential face of the power spiral tube 132 so as to extend along the longitudinal axis of the power spiral tube 132. The power spiral tube 132 may be configured to be detachable from the rotating section 130.

The power spiral tube 132 is connected to a motor 150 provided as an actuator in the control unit 160. The motor 150 is connected to the controller 200 through a signal line for actuator current signals (not shown in the drawings) passing through the control unit 160 and the universal cable 190.

The motor 150 is operated by a manipulation using the foot switch 360. The rotational force of the motor 150 is transmitted to the rotating section 130. Thereby, the fin 134 provided on the power spiral tube 132 rotates around the longitudinal axis.

When the fin 134 rotates in contact with a wall, such as an inner wall of a body cavity, a propulsion force that propels the insertion section 110 by itself is generated. In the small intestine or the large intestine, for example, the fin 134 crawls along the folds on the inner wall of the small intestine or the large intestine, and thereby a propulsion force acts on the insertion section 110. By this propulsion force, the insertion section 110 is propelled by itself. The self-propulsion of the insertion section 110 assists in the tasks of insertion and removal of the insertion section 110 by the user. In the explanation that follows, the direction of rotation of the motor 150 that is propelled by itself toward the distal side of the insertion section 110 will be referred to as a normal direction (insertion direction), and the direction of rotation of the motor 150 that is propelled by itself toward the proximal side of the insertion section 110 will be referred to as a reverse direction (removal direction).

The motor 150 is provided with an encoder 152. The encoder 152 generates a rotation speed signal according to the rotation speed of the motor 150. The rotation speed signal is transmitted to the controller 200 through a rotation speed signal line (not shown in the drawings) passing through the universal cable 190.

The image observation monitor 310 includes a general display apparatus such as a liquid crystal display. The image observation monitor 310 displays an endoscopic image based on an image signal obtained by, for example, the imaging element 120.

The foot switch 360 includes a forward pedal 362 and a backward pedal 364. The forward pedal 362 is a pedal depressed by the user when the user wants to make the motor 150 rotate in the normal direction. The backward pedal 364 is a pedal depressed by the user when the user wants to make the motor 150 rotate in the reverse direction. Each of the forward pedal 362 and the backward pedal 364 is configured such that the amount of depression is detected.

The controller 200 controls the operation of the self-propelled mechanism 101. The controller 200 includes a rotation speed acquisition section 201, a motor driving circuit 202, a torque acquisition section 203, a recording section 204, and a control section 210.

The rotation speed acquisition section 201 acquires a rotation speed signal input from the encoder 152 at every predetermined sampling interval. The rotation speed acquisition section 201 acquires a speed value corresponding to the rotation speed of the motor 150 on the basis of the acquired rotation speed signal, and transmits the speed value to the control section 210.

The motor driving circuit 202 is configured by, for example, a driver amplification circuit. The motor driving circuit 202 drives the motor 150 on the basis of the instruction value. Thereby, the motor 150 rotates in the normal direction to generate a rotation speed or a torque corresponding to the amount of depression of the forward pedal 362. Also, the motor 150 rotates in the reverse direction to generate a rotation speed or a torque corresponding to the amount of depression of the backward pedal 364.

The torque acquisition section 203 acquires a current value of a motor current output from the motor driving circuit 202. The torque acquisition section 203 transmits, as a torque value, a signal corresponding to the magnitude of the motor current, namely, a signal corresponding to the torque of the motor 150 to the control section 210.

The recording section 204 is a recording medium that retains its contents with the power turned off, such as a flash memory, and records data such as programs for operating the controller 200 and various set values. The recording section 204 is not limited to a semiconductor memory, and may be, for example, a magnetic or optical medium. That is, each recording medium may bear the function of the recording section 204 as a recording section.

The control section 210 includes a driving control section 211, a mode switching section 212, a threshold determination section 213, and an input determination section 214.

The driving control section 211 sets the control mode of the motor 150, and controls operation of the motor driving circuit 202. The driving control section 211 includes a speed control section 221 and a torque control section 222. The speed control section 221 controls the operation of the motor driving circuit 202 based on the speed control mode, to adjust the rotation speed of the motor 150 to a predetermined value. On the other hand, the torque control section 222 adjusts the torque generated by the motor 150 by controlling the operation of the motor driving circuit 202 based on the torque control mode.

The mode switching section 212 determines whether to cause the driving control section 211 to perform control based on the speed control mode or the torque control mode, and instructs the driving control section 211 of the control mode, on the basis of predetermined switching conditions.

The threshold determination section 213 compares the rotation speed acquired by the rotation speed acquisition section 201 or the torque acquired by the torque acquisition section 203 with a predetermined threshold value, and transmits the compared result to the driving control section 211. The driving control section 211 changes control on the basis of the compared result acquired from the threshold determination section 213.

The input determination section 214 determines, for example, input to the foot switch 360. The input determination section 214 determines whether the forward pedal 362 or the backward pedal 364 is depressed, and, if either of the pedals is depressed, determines the amount of depression.

The image processor 280 includes a light source section 281 and an image processing section 282. The light source section 281 includes, for example, a white LED or a xenon lamp, and inputs light to an optical fiber (not shown in the drawings) in the universal cable 190. This light is emitted from the illumination lens 121.

The image processing section 282 acquires an image signal from the imaging element 120 through the insertion section 110 and the universal cable 190. The image processing section 282 performs image processing on the acquired image signal. Also, the image processing section 282 transmits the processed image signal to the image observation monitor 310, and causes the image observation monitor 310 to display an endoscopic image.

The control section 210 may include, for example, a central processing unit (CPU), an application specific integrated circuit (ASIC), or a field-programmable gate array (FPGA). Each section of the controller 200 may be formed of, for example, one integrated circuit, or may be formed of a combination of integrated circuits. The operation of the controller 210 is executed in accordance with a program recorded in, for example, a recording area in the control section 210 or the recording section 204.

<Operation of Insertion Apparatus>

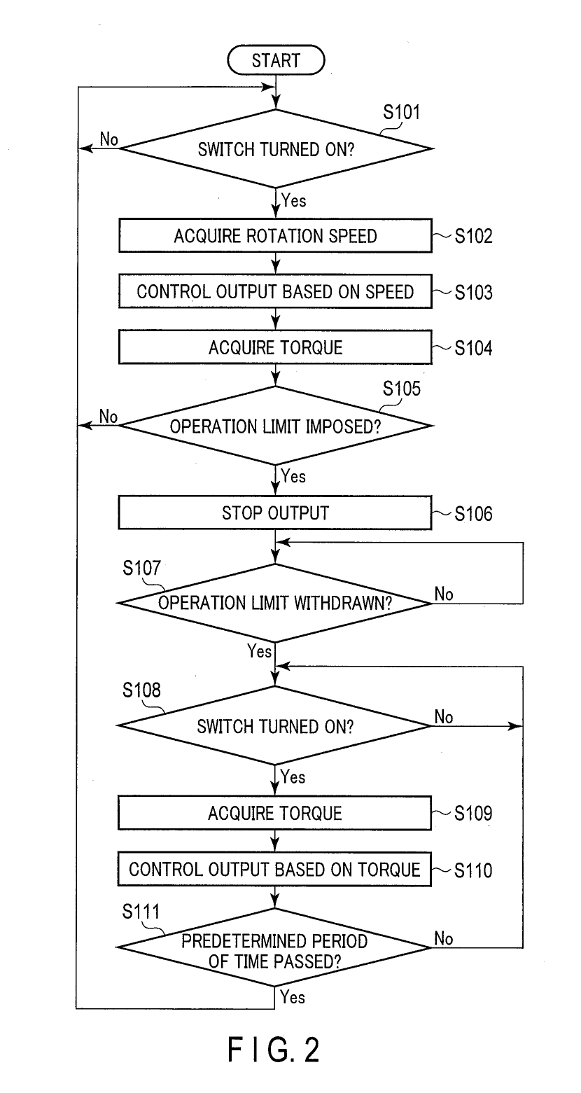

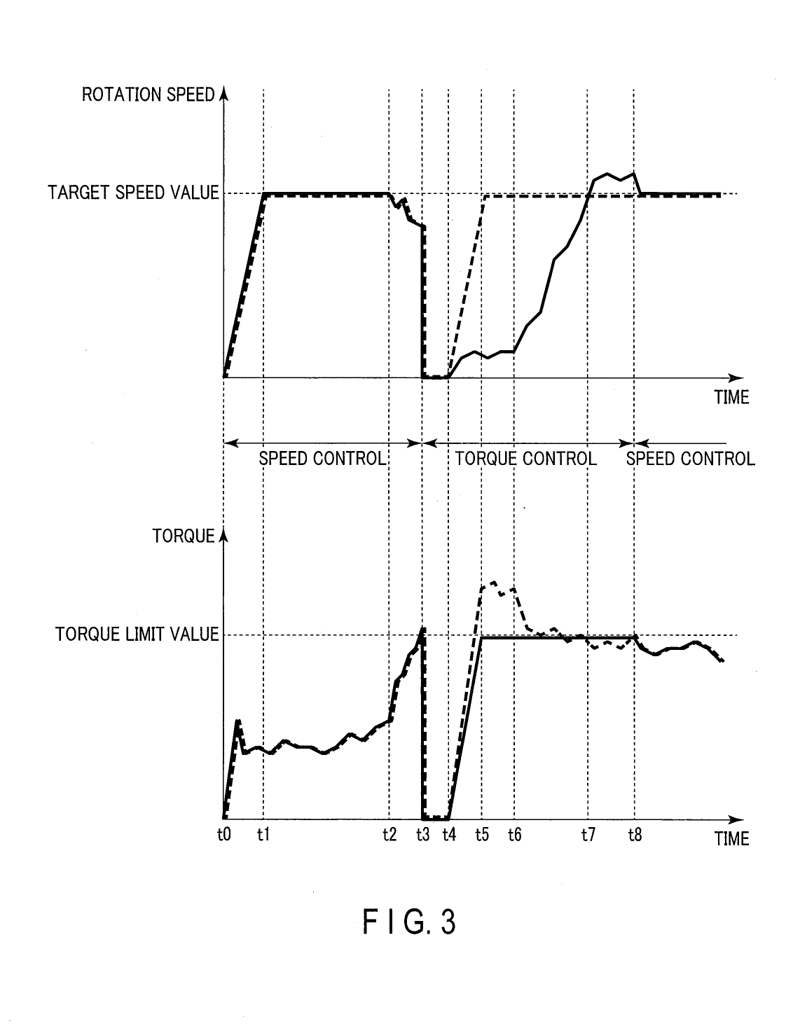

The operation of the insertion apparatus 1 according to the present embodiment will be described with reference to FIGS. 2 and 3. FIG. 2 is a flowchart schematically showing an example of processing performed in the controller. FIG. 3 is a diagram showing an example of a relation between a rotation speed and a torque value of the motor 150 with respect to the passage of time. In FIG. 3, the top part represents changes in rotation speed with respect to time, and the bottom part represents changes in torque value with respect to time. In each of the drawings, the solid line represents an example of an operation according to the present embodiment, and the dashed line represents an example of an operation according to a comparative example. Here, a description will be made on the operation of the insertion apparatus 1, with respect to the case, as an example, where the switch of the foot switch 360 is depressed to the maximum extent, without distinction between forward and backward. More precisely, control is performed as to whether rotation is performed in the insertion direction or in the removal direction, according to whether the forward pedal 362 is depressed or the backward pedal 364 is depressed. Also, the target value, which will be described later, is changed according to the amount of depression of the foot switch 360.

In step S101, an input determination section 214 of the control section 210 of the controller 200 determines whether or not the foot switch 360 is depressed and turned on. If the foot switch 360 is not turned on, the processing stands by, repeating step S101. If the foot switch 360 is turned on, the driving control section 211 of the control section 210 starts rotating the motor 150, and the processing advances to step S102.

In step S102, the rotation speed acquisition section 201 of the controller 200 acquires the rotation speed of the motor 150. In step S103, the driving control section 211 of the control section 210 of the controller 200 controls output of the motor driving circuit 202 by providing feedback to maintain the rotation speed at a predetermined target value. That is, the driving control section 211 controls output of the motor driving circuit 202 based on the speed control mode.

In step S104, the torque acquisition section 203 of the controller 200 acquires the value of the torque generated by the motor 150 on the basis of the current value output from the motor driving circuit 202. In step S105, the threshold determination section 213 of the controller 200 determines whether or not an operation limit should be imposed, based on whether or not the acquired torque value has exceeded a predetermined threshold value. If the torque value does not exceed the threshold value and the operation limit is not to be imposed, the processing returns to step S101. That is, control is performed based on the speed control mode until the operation limit is imposed.

Let us assume that, in the example shown in FIG. 3, the foot switch 360 is turned on at time t0. At this time, the driving control section 211 performs control to maintain the rotation speed at the target speed value. As shown in the top part of FIG. 3, the rotation speed of the motor 150 does not suddenly reach the target speed value; instead, the rotation speed gradually increases from time t0 to time t1. Let us assume that the rotation speed reaches the target speed value at time t1. Thereafter, the output is controlled in such a manner that the rotation speed remains constant at the target speed value from time t1 to time t2, as shown. At this time, the torque generated by the motor 150 is illustrated in the bottom part. That is, the torque value generated by the motor 150 changes according to the rotation speed of the self-propelled mechanism 101 and the surrounding situation of the self-propelled mechanism 101. For example, when the rotation of the power spiral tube 132 of the self-propelled mechanism 101 is not prevented, the torque value required for rotation at the rotation speed with the target speed value is relatively small. On the other hand, when the rotation of the power spiral tube 132 is prevented, the torque value required for rotation at the rotation speed with the target speed value becomes relatively large. When the rotation starts, for example, the resistance inside or outside the self-propelled mechanism 101 causes the torque value to momentarily increase. Thereafter, the torque value changes according to the surrounding environment.

If it is determined in step S105 that the operation limit should be imposed, the processing advances to step S106. In step S106, the driving control section 211 of the controller 200 restricts the operation of the motor 150. That is, the driving control section 211 causes the motor driving circuit 202 to stop output, and causes the motor 150 to stop rotating. In this manner, the operation limit functions as a torque limit.

For example, when the insertion section 110 gets stuck in a tract, the power spiral tube 132 is prevented from rotating smoothly, and the rotation speed decreases from time t2 to time t3, as shown. At this time, the torque value increases. Let us assume that the torque value has exceeded a torque limit value at time t3. At this time, the rotation of the motor 150 is stopped. As a result, at time t3, the rotation is stopped, the rotation speed becomes zero, and a torque is not generated, as shown in FIG. 3.

In step S107, the input determination section 214 of the controller 200 determines whether or not a manipulation to withdraw the operation limit has been performed. The manipulation to withdraw the operation limit includes, but not limited to, for example, releasing depression of the foot switch 360, depressing one of the forward pedal 362 and the backward pedal 364 that is opposite to the currently depressed pedal, depressing a dedicated button to withdraw the operation limit, etc. If a manipulation to withdraw the operation limit is not performed, the processing stands by, repeating step S107. On the other hand, if a manipulation to withdraw the operation limit is performed, the processing advances to step S108.

In step S108, an input determination section 214 of the controller 200 determines whether or not the foot switch 360 is turned on. If the foot switch 360 is not turned on, the processing stands by, repeating step S108. If the foot switch 360 is turned on, the driving control section 211 of the control section 210 starts rotating the motor 150, and the processing advances to step S109.

In step S109, the torque acquisition section 203 of the controller 200 acquires a torque value generated in the motor 150. In step S110, the driving control section 211 of the control section 210 of the controller 200 resumes controlling output of the motor driving circuit 202, using the torque control section 222.

In step S111, the threshold determination section 213 of the controller 200 determines whether or not a predetermined period of time has passed since the output is stopped in step S106. If it is determined that the predetermined period of time has not passed, the processing returns to step S108. That is, the control based on the torque control mode is continued until the predetermined period of time passes.

Let us assume that, in the example shown in FIG. 3, the foot switch 360 is turned on at time t4. If the foot switch 360 is depressed, the value of the torque generated in the motor is controlled by setting a value slightly lower than the torque limit value as a target value, as shown in the bottom part of FIG. 3. The torque value gradually increases from time t4 to time t5, and the torque value is maintained at a target value slightly lower than the torque limit value from time t5 to time t8. At this time, the rotation speed gradually increases according to increase in torque value, as shown by the top part of FIG. 3. When the cause of the insertion section 110 being stuck in the tract is removed, the rotation speed increases at time t6 and thereafter, as shown, thus allowing the insertion section 110 to move forward smoothly. At this time, the rotation speed may exceed the target speed value of when the speed control mode is used from time t7 to time t8, as shown. The rotation speed may be suitably limited.

If it is determined in step S111 that the predetermined period of time has passed, the processing returns to step S101. That is, the operation from step S101 is repeated, and control of the rotation speed of the motor 150 is performed based on the speed control mode.

In the example shown in FIG. 3, the period from time t3 to time t8 is the predetermined period of time during which determination is performed in step S111. At time t8, after the predetermined period of time has passed since the control based on the torque control mode is started, the control mode is changed to the speed control mode. Accordingly, at time t8 and thereafter, control is performed to maintain the rotation speed at the target speed value. At this time, the torque value is measured according to the surrounding situation of the insertion section 110. The time until the change is made to the speed control mode may be based on the amount of time that has passed since the rotation of the motor 150 is resumed after the rotation is stopped, instead of the amount of time that has passed since the motor 150 is stopped.

As a comparative example, changes in rotation speed and torque value in the case where only the speed control mode is used and the torque control mode is not used are shown by dashed lines in FIG. 3. Let us assume that, as shown by the top part of FIG. 3, an operation limit is imposed at time t3, when the insertion section 110 gets stuck in a tract, for example, and the foot switch 360 is turned on again at time t4. At this time, when control is performed based on the speed control mode, since the insertion section 110 is stuck in the tract, the torque value becomes higher than the torque limit value again after time t4 and thereafter, as shown by the bottom part of FIG. 3. If an operation limit were not imposed, the torque value would be higher than the torque limit value until the cause of the insertion section being stuck is removed. It is not preferable that the torque becomes too high. If an operation limit were imposed when the torque has exceeded the torque limit value, the operation limit would be repeatedly imposed. In this case, the self-propelled mechanism 101 cannot be substantially utilized until the cause of the insertion section being stuck is removed.

According to the present embodiment, since the speed control mode is used in a normal state, it is possible for the user to adjust the insertion section 110 to move forward or backward as desired, based on depression of the foot switch 360. On the other hand, if the torque has become too high, the operation is temporarily stopped, and the control mode is switched to the torque control mode. It is thereby possible to continue operation of the self-propelled mechanism 101, while suppressing generation of a torque within a predetermined range. Thereafter, when a predetermined period of time has passed, the control mode is switched again to the speed control mode, which allows the user to perform a manipulation easier. By these operations, it is possible to achieve both ease of manipulation and suppression of torque within a predetermined range.

<Modification>

The self-propelled mechanism may have any configuration. For example, a configuration may be adopted in which a belt configured to rotate in the longitudinal direction of the insertion section 110 is provided along the outer periphery of the insertion section 110.

### Second Embodiment

A second embodiment will be described. Herein, differences from the first embodiment will be described. The same symbols will be used to denote similar structural elements, and a description of such structural elements will be omitted. In the present embodiment, the method of controlling operation of the self-propelled mechanism 101 is different from that of the first embodiment. In the present embodiment, if the torque value becomes higher than a predetermined threshold torque value while the speed control mode is used, the control mode is switched to the torque control mode. If the rotation speed becomes higher than a predetermined threshold speed value while the torque control mode is used, the control mode is switched to the speed control mode.

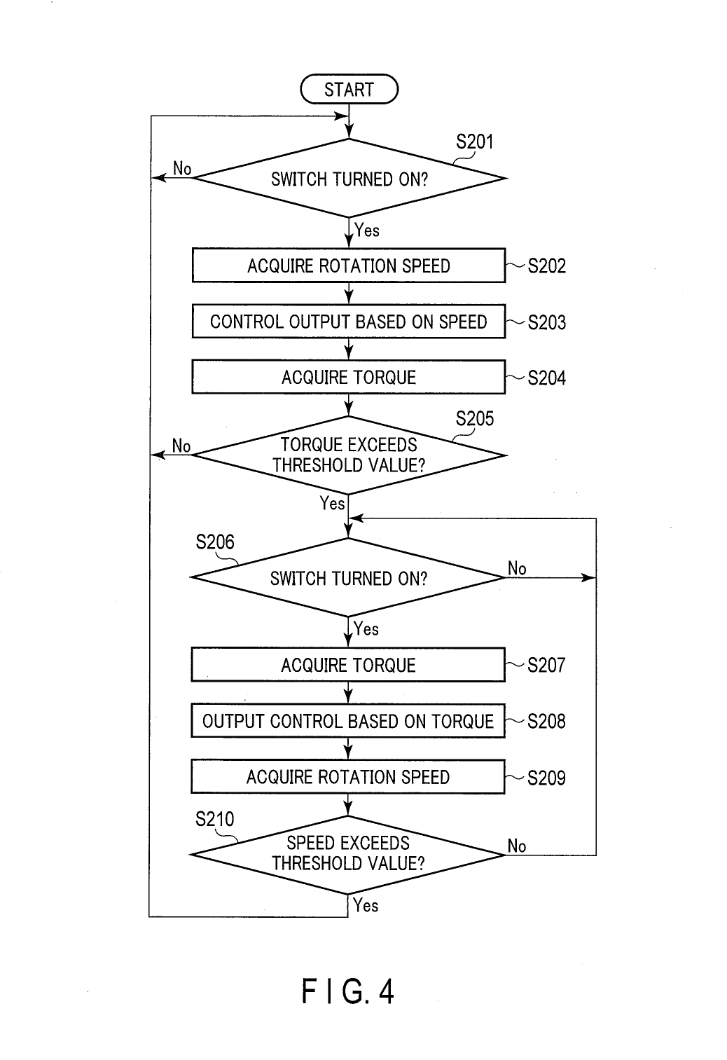

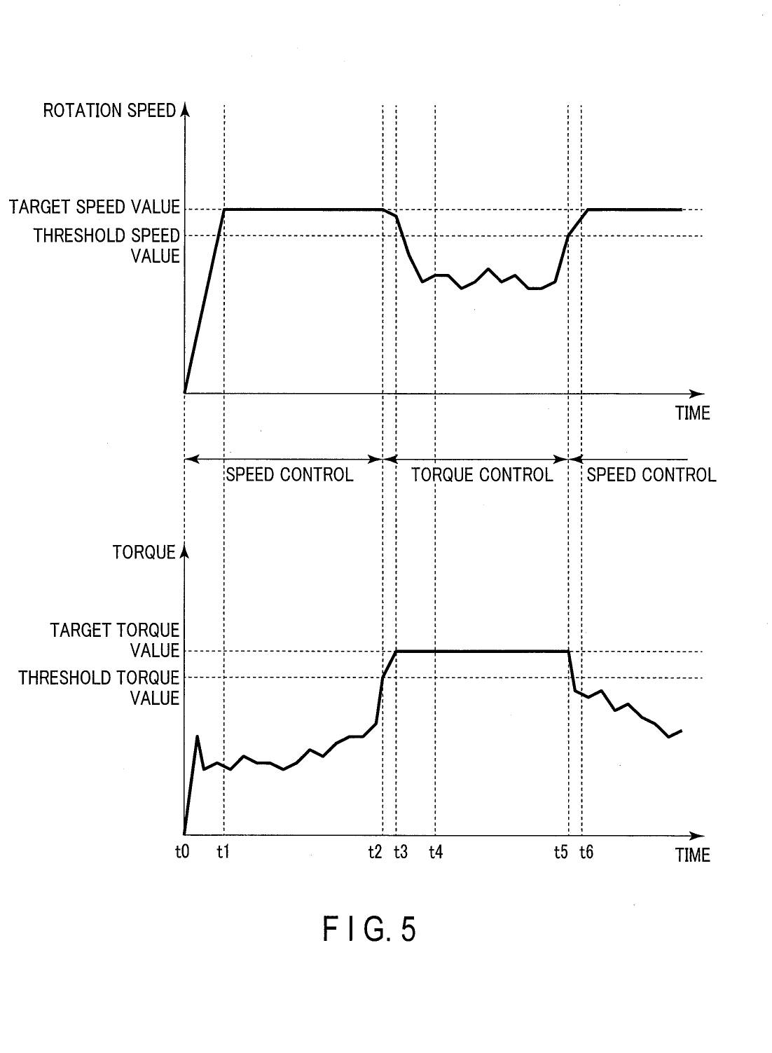

The operation of the insertion apparatus 1 according to the present embodiment will be described with reference to FIGS. 4 and 5. FIG. 4 is a flowchart schematically showing an example of processing performed in the controller. FIG. 5 is a diagram showing an example of a relation between a rotation speed and a torque value with respect to the passage of time. In FIG. 5, the top part represents changes in rotation speed with respect to time, and the bottom part represents changes in torque value with respect to time. Here, a description will be made on the operation of the insertion apparatus 1, with respect to the case, as an example, where the switch of the foot switch 360 is depressed to the maximum extent, without distinction between forward and backward.

In step S201, the input determination section 214 of the control section 210 of the controller 200 determines whether or not the foot switch 360 is turned on. If the foot switch 360 is not turned on, the processing stands by, repeating step S201. If the foot switch 360 is turned on, the driving control section 211 of the control section 210 starts rotating the motor 150, and the processing advances to step S202.

In step S202, the rotation speed acquisition section 201 of the controller 200 acquires the rotation speed of the motor 150. In step S203, the driving control section 211 of the control section 210 of the controller 200 controls output of the motor driving circuit 202 by performing feedback to maintain the rotation speed at a predetermined target value. That is, the driving control section 211 controls output of the motor driving circuit 202 based on the speed control mode.

In step S204, the torque acquisition section 203 of the controller 200 acquires the value of the torque generated by the motor 150 on the basis of the current value output from the motor driving circuit 202. In step S205, the threshold determination section 213 of the controller 200 determines whether or not the acquired torque value has exceeded a predetermined threshold torque value. If the torque value does not exceed the threshold torque value, the processing returns to step S201. That is, if the torque value is lower than the threshold torque value, control is performed based on the speed control mode.

In step S205, if it is determined that the torque value is higher than the predetermined threshold torque value, the processing advances to step S206. In step S206, the input determination section 214 of the controller 200 determines whether or not the foot switch 360 is turned on. If the foot switch 360 is not turned on, the motor 150 is stopped, and the processing stands by, repeating step S206. If the foot switch 360 is turned on, the driving control section 211 of the control section 210 causes the motor 150 to rotate, and the processing advances to step S207.

In step S207, the torque acquisition section 203 of the controller 200 acquires the value of the torque generated in the motor 150. In step S208, the driving control section 211 of the control section 210 of the controller 200 controls output of the motor driving circuit 202, using the torque control section 222. That is, the driving control section 211 controls output of the motor driving circuit 202 based on the torque control mode.

In step S209, the rotation speed acquisition section 201 of the controller 200 acquires the rotation speed of the motor 150. In step S210, the threshold determination section 213 of the controller 200 determines whether or not the acquired rotation speed exceeds a predetermined threshold speed value. If the rotation speed is not higher than the threshold speed value, the processing returns to step S206. That is, the control based on the torque control mode is continued if the rotation speed is lower than the threshold speed value. If the rotation speed is higher than the threshold speed value, the processing returns to step S201. That is, if the rotation speed has become higher than the threshold speed value, the control mode is switched to the speed control mode.

An example of the operation according to the present embodiment will be described with reference to FIG. 5. Let us assume that the foot switch 360 is turned on at time t0. At this time, since control is performed based on the speed control mode, the rotation speed is controlled in such a manner that the rotation speed gradually increases and is maintained at the target speed value at time t1 and thereafter. At this time, the torque generated in the motor 150 changes according to the surrounding environment, etc., of the insertion section 110. For example, the torque value increases when the insertion section 110 gets stuck in a tract.

In the example shown in FIG. 5, the torque value exceeds the threshold torque value at time t2. Accordingly, the control mode is switched to the torque control mode at time t2. That is, control is performed to maintain the torque value at the target torque value. In the example shown in FIG. 5, control is performed to maintain the torque value at the target torque value at time t3 and thereafter. At this time, the rotation speed changes according to the surrounding environment of the insertion section 110. For example, when the insertion section 110 gets stuck in a tract, the rotation speed becomes a value lower than the target speed value.

Thereafter, when the cause of the insertion section 110 being stuck is removed, the rotation speed increases. In the example shown in FIG. 5, the rotation speed exceeds the threshold speed value at time t5. At time t5, the control mode is changed from the torque control mode to the speed control mode. Accordingly, control is performed to maintain the rotation speed at the target speed value thereafter. At this time, the torque value becomes a value that is adjusted according to the surrounding environment. For example, when the insertion section 110 is not stuck in a tract, the torque value decreases to a low value.

Typically, the rotation speed increases and the torque value decreases when the resistance of the rotation of the power spiral tube 132 is low, whereas the rotation speed decreases and the torque value increases when the resistance is high. In the present embodiment, the speed control mode is preferentially adopted to allow the user to easily adjust the operation of the self-propelled mechanism 101, whereas the torque control mode is used when the torque value is high, to prevent the torque value from becoming too high. As a result, it is possible to achieve both ease of manipulation of the self-propelled mechanism 101 and suppression of the torque value within a predetermined range.

<Modification>

Regarding the output control of the self-propelled mechanism 101, the control mode has been described, in both of the first embodiment and the second embodiment, as being switched from the speed control mode to the torque control mode when the torque value has exceeded a predetermined threshold value. Regarding the change of the control mode from the torque control mode to the speed control mode, an example has been described in the first embodiment in which a change is made to the speed control mode when a predetermined period of time has passed, and an example has been described in the second embodiment in which a change is made to the speed control mode when the rotation speed has exceeded a predetermined threshold value. The determination to change the control mode from the torque control mode to the speed control mode is not limited thereto. For example, when the rotation speed has exceeded a predetermined threshold value in the first embodiment, the control mode may be changed to the speed control mode, and when a predetermined period of time has passed in the second embodiment, the control mode may be changed to the speed control mode. Moreover, when the rotation direction has been changed between the insertion direction and the removal direction, for example, in either of the embodiments, the control mode may be changed to the speed control mode. Furthermore, an input section such as a button, to which an input to switch the control mode is made, may be provided in such a manner that the control mode is changed to the speed control mode based on the input to the input section.

In the above-described example, a case has been described as an example where the control mode is switched between the speed control mode and the torque control mode; however, the control mode is not limited thereto. In addition to the speed control mode and the torque control mode, another control mode, such as a voltage control mode, which performs control to maintain the voltage to be supplied to the motor 150 at a target value, may be used.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A controller configured to control an operation of a self-propelled mechanism provided in an elongated insertion section of an endoscope, the self-propelled mechanism including a motor configured to generate a force that causes the insertion section to be inserted into or removed from a subject, the controller comprising:
an input device to which an input is provided to switch a control mode; and
at least one circuit configured to:
acquire a speed value corresponding to a rotation speed of the motor;
acquire a torque value corresponding to a torque generated by the motor;
control rotation of the motor by switching the control mode, the control mode including at least two of speed control that controls rotation of the motor based on the speed value and torque control that controls rotation of the motor based on the torque value; and
stop the rotation of the motor if the torque value has exceeded a predetermined threshold value, perform the torque control when the rotation of the motor is resumed, and change the control mode from the torque control to the speed control when the input is provided after the control mode has been changed from the speed control to the torque control.

2. An insertion apparatus comprising:
the controller according to claim 1; and
the endoscope.

3. The insertion apparatus according to claim 2, wherein the self-propelled mechanism comprises:
a rotating cylindrical body provided on an outer circumferential face of the insertion section so as to be rotatable around a longitudinal axis of the insertion section;
the motor configured to rotate the rotating cylindrical body; and
a fin formed in a spiral shape on an outer circumferential face of the rotating cylindrical body and configured to cause the insertion section to be inserted or removed in accordance with the rotation of the rotating cylindrical body.

4. A controller configured to control an operation of a self-propelled mechanism provided in an elongated insertion section of an endoscope, the self-propelled mechanism including a motor configured to generate a force that causes the insertion section to be inserted into or removed from a subject, the controller comprising:
an input device to which a first input to cause the motor to rotate in a normal direction and a second input to cause the motor to rotate in a reverse direction are provided,
at least one circuit configured to:
acquire a speed value corresponding to a rotation speed of the motor;
acquire a torque value corresponding to a torque generated by the motor;
control rotation of the motor by switching the control mode, the control mode including at least two of speed control that controls rotation of the motor based on the speed value and torque control that controls rotation of the motor based on the torque value; and
stop the rotation of the motor if the torque value has exceeded a predetermined threshold value, perform the torque control when the rotation of the motor is resumed, and change the control mode from the torque control to the speed control when one of the first input and the second input that is different from an input provided when the control mode has been changed from the speed control to the torque control is provided.

5. An insertion apparatus comprising:
the controller according to claim 4; and
the endoscope.

6. The insertion apparatus according to claim 5, wherein the self-propelled mechanism comprises:
a rotating cylindrical body provided on an outer circumferential face of the insertion section so as to be rotatable around a longitudinal axis of the insertion section;
a motor configured to rotate the rotating cylindrical body; and
a fin formed in a spiral shape on an outer circumferential face of the rotating cylindrical body and configured to cause the insertion section to be inserted or removed in accordance with the rotation of the rotating cylindrical body.

7. A controller configured to control an operation of a self-propelled mechanism provided in an elongated insertion section of an endoscope, the self-propelled mechanism including a motor configured to generate a force that causes the insertion section to be inserted into or removed from a subject, the controller comprising:
at least one circuit configured to:
acquire a speed value corresponding to a rotation speed of the motor;
acquire a torque value corresponding to a torque generated by the motor;
control rotation of the motor by switching the control mode, the control mode including at least two of speed control that controls rotation of the motor based on the speed value and torque control that controls rotation of the motor based on the torque value; and
stop the rotation of the motor if the torque value has exceeded a predetermined threshold value, perform the torque control when the rotation of the motor is resumed, and monitor the speed value to change the control mode from the torque control to the speed control after the speed value has exceeded a predetermined threshold value.

8. An insertion apparatus comprising:
the controller according to claim 7, and
the endoscope.

9. The insertion apparatus according to claim 8, wherein the self-propelled mechanism comprises:
a rotating cylindrical body provided on an outer circumferential face of the insertion section so as to be rotatable around a longitudinal axis of the insertion section;
a motor configured to rotate the rotating cylindrical body; and
a fin formed in a spiral shape on an outer circumferential face of the rotating cylindrical body and configured to cause the insertion section to be inserted or removed in accordance with the rotation of the rotating cylindrical body.
